(19) United States
(12) Patent Application Publication
(10) Pub. No.: US 2014/0200578 A1
(43) Pub. Date: Jul. 17, 2014

(54) RENAL NERVE ABLATION CATHETER

(71) Applicant: BOSTON SCIENTIFIC SCIMED, INC., Maple Grove, MN (US)

(72) Inventors: JOEL N. GROFF, MONTROSE, MN (US); MARK L. JENSON, GREENFIELD, MN (US); MARTIN R. WILLARD, BURNSVILLE, MN (US); JASON P. HILL, BROOKLYN PARK, MN (US); GARY L. HENDRICKSON, BIG LAKE, MN (US)

(73) Assignee: BOSTON SCIENTIFIC SCIMED, INC., MAPLE GROVE, MN (US)

(21) Appl. No.: 14/155,028

(22) Filed: Jan. 14, 2014

Related U.S. Application Data
(60) Provisional application No. 61/752,217, filed on Jan. 14, 2013.

(51) Int. Cl. A61B 18/14

(52) U.S. Cl. CPC A61B 18/1492
USPC 606/41

(57) ABSTRACT

Medical devices for ablating nerves perivascularly and methods for making and using the same are disclosed. An example medical device may include an expandable frame slidably disposed within a catheter shaft. The expandable frame may be configured to shift between a collapsed configuration and an expanded configuration. One or more electrodes may be disposed on a surface of the expandable frame. The one or more electrodes may be disposed radially inward relative to the greatest radial extent of the expandable frame when the expandable frame is in the expanded configuration.

### CROSS-REFERENCE TO RELATED APPLICATIONS

This application claims priority under 35 U.S.C. §119 to U.S. Provisional Application Ser. No. 61/752,217, filed Jan. 14, 2013, the entirety of which is incorporated herein by reference.

### TECHNICAL FIELD

The present disclosure pertains to medical devices, and methods for manufacturing medical devices. More particularly, the present disclosure pertains to ablating and/or modulating renal nerves.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include an expandable frame slidably disposed within a catheter shaft. The expandable frame may be configured to shift between a collapsed configuration and an expanded configuration. One or more electrodes may be disposed on a surface of the expandable frame. The one or more electrodes may be disposed radially inward relative to the greatest radial extent of the expandable frame when the expandable frame is in the expanded configuration.

Another example medical apparatus for ablating renal nerves perivascularly may include a catheter shaft having a proximal end, a distal end, and a lumen extending from the proximal to the distal end. An expandable member may be slidably disposed within the catheter shaft. Additionally, the apparatus may include a control mechanism to control the expansion and contraction of the expandable member, wherein the expandable member may include one or more electrodes disposed on a surface of the expandable member configured radially inwards relative to the greatest radial extent of the expandable member.

A method for ablating nerves perivascularly may include providing a medical device. The medical device may include an expandable frame slidably disposed within a catheter shaft. The expandable frame may be configured to shift between a collapsed configuration and an expanded configuration. One or more electrodes may be disposed on a surface of the expandable frame. The one or more electrodes may be disposed radially inward relative to the greatest radial extent of the expandable frame when the expandable frame is in the expanded configuration. The method may also include advancing the medical device through a body lumen to a position adjacent to an area of interest, shifting the expandable frame from the collapsed configuration to the expanded configuration, and activating at least some of the one or more electrodes.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view illustrating an example renal nerve modulation system.

FIGS. 2A-2C illustrate the distal portion of the renal nerve modulation system according to the present disclosure, located within a renal artery.

FIG. 3 depicts an alternate embodiment of an expandable member.

FIGS. 4-6B illustrate another alternate embodiment of the expandable member.

FIG. 7 illustrates another alternate embodiment of the expandable member.

FIG. 8 illustrates another alternate embodiment of the expandable member.

FIG. 9 illustrates another alternate embodiment of the expandable member.

FIGS. 10A-10C depict variations in the expandable member shown in FIG. 9.

FIGS. 11A-11E depict different insulation configuration of ribbons forming the expandable member.

FIG. 12 illustrates an alternate embodiment of the ribbon.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

Certain treatments may require the temporary or permanent interruption or modification of select nerve function. One example treatment is renal nerve ablation which is sometimes used to treat conditions related to hypertension. The kidneys produce a sympathetic response, which, among other effects, increases the undesired retention of water and/or sodium. Ablating some of the nerves running to the kidneys may reduce or eliminate this sympathetic function, which may provide a corresponding reduction in the associated undesired symptoms (e.g., high blood pressure).

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

Because the nerves are hard to visualize, treatment methods employing such energy sources have tended to apply the energy as a generally circumferential ring or helix to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

While the devices and methods described herein are discussed relative to renal nerve modulation through a blood vessel wall, it is contemplated that the devices and methods may be used in other applications where nerve modulation and/or ablation are desired. The term modulation refers to ablation and other techniques that may alter the function of affected nerves.

Renal nerve ablation may require precise control of the catheter during treatment. Once the desired position is achieved, the operator must maintain that position stably during ablation. Afterward, the catheter can be repositioned for additional ablation, if desired. Catheter control may be enhanced by tactile feedback, to help the user apply appropriate force between the catheter and the surrounding tissue. Tactile feedback takes advantage of the user's sense of touch by relaying forces to the user.

Some embodiments of the present disclosure include a medical device for ablating a target tissue within a patient's body. The medical device may take the form of a catheter having an expandable member slidably disposed within its distal portion. The catheter may be configured to ablate a desired body tissue by, for example, applying energy (e.g., RF energy). The expandable member may include electrodes circumferentially disposed over a portion thereof. The electrodes may be disposed at locations that are positioned radially inward relative to the greatest radial extent of the expandable member. In other words, the electrodes may be positioned at locations offset from the widest portion of the expandable member (when in the expandable member is expanded). This may include positions that are longitudinally shifted from the widest portion of the expanded expandable member. Upon expansion of the expandable member within a blood vessel, the electrodes may be placed at a location proximal to but not contacting the wall of the blood vessel. This placement of electrodes may aid in conducting off-wall renal nerve ablation. The electrodes may be used in various combinations to conduct ablation and may have various shapes and sizes to define the heating geometry of the vessel during ablation.

The ablation catheter of the present disclosure may be employed to modulate and/or ablate one or more renal nerves. It will be understood that this application is merely exemplary, and that the catheter of the present disclosure may be used in any desired body part requiring ablation without departing from the scope of the present disclosure.

For purposes of this disclosure, “proximal” refers to the end or direction closer to the operator during use, and “distal” refers to the end or direction further from the operator during use.

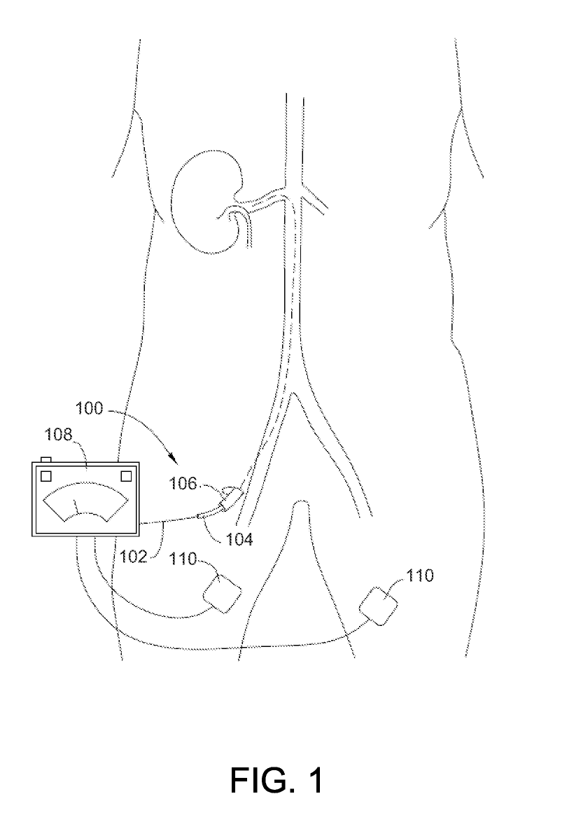

FIG. 1 is a schematic view of an illustrative renal nerve modulation system 100 in situ. System 100 may include one or more conductive element(s) 102 providing power to renal ablation system 104 disposed within a guide catheter 106. A proximal end of conductive element 102 may be connected to a control and power element 108, which supplies the necessary electrical energy to activate one or more electrodes at or near a distal end of the renal ablation system 104. In some instances, ground pads 110 may be supplied on the legs or at another conventional location on the patient's body to complete the circuit. The control and power element 108 may include monitoring elements to monitor parameters such as power, temperature, current, impedance, voltage, pulse size and/or shape and other suitable parameters as well as suitable controls for performing the desired procedure. The control and power element 108 may control radio frequency (RF) electrodes, which may be configured to operate at a frequency of approximately 460 kHz. It is contemplated that any desired frequency in the RF range may be used, for example, from 450-500 kHz. It is, however, contemplated that different types of energy outside the RF spectrum may be used as desired, for example, but not limited to ultrasound, microwave, and laser.

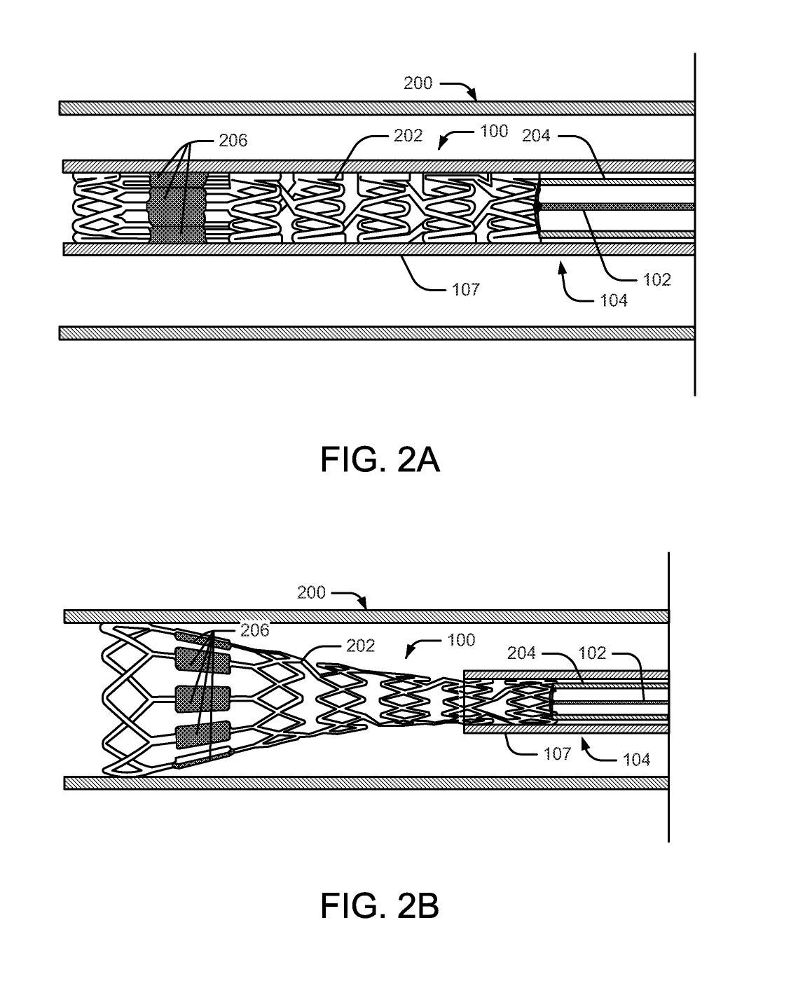

FIGS. 2A-2C illustrate the distal portion of the renal nerve modulation system 100 located within a blood vessel 200. FIG. 2A illustrates the system 100 in a retracted position. FIG. 2B depicts the system 100 in a deployed position, and FIG. 2C shows system 100 in operation. As shown, the renal ablation system 104 may include an outer sheath 107 that may be configured to shift between retracted and the deployed positions. The renal ablation system 104 may also include an inner shaft 204 disposed within the outer sheath 107. The outer sheath 107 may be slidable relative to the inner shaft 204. An expandable frame or member 202 may be attached to or otherwise disposed at the distal end of an inner shaft 204. A plurality of electrodes 206 may be disposed circumferentially on a portion of the expandable member 202 with electrodes 206 positioned radially inwards relative to the greatest radial extent of the expandable member 202. In this example, the electrodes 206 are positioned proximally of the distal end of the expandable member 202 such that the electrodes 206 are positioned radially inward relative to the widest point of the expandable member 202.

The conductive element 102 may be coupled to electrodes 206. In some embodiments, the conductive element 102 may pass through the lumen of the inner shaft 204, and may communicate with the electrodes 206 by a connection through the frame of the expandable member 202. Other configurations are contemplated. The circumferentially placed electrodes 206 may simplify and increase the speed of renal nerve ablation, as this circumferential placement may reduce the need for repeated RF ablation and electrode repositioning (which may increase the reliability of an ablation procedure and may reduce the total procedure time).

Outer sheath 107 and inner shaft 204 may be tubular members of suitable length and cross-sectional dimensions. They may be dimensioned to facilitate introduction of the system 100 within the desired blood vessel 200. Thus, a particular outer sheath 107 may be chosen to have an outside diameter less than that of blood vessel 200. Similarly, the diameter of inner shaft 204 may be less than that of outer sheath 107 to be slidably disposed within outer sheath 107. The length of outer sheath 107 and the inner shaft 204 may depend upon the location of blood vessel 200 within a patient's body. In other embodiments, the inner shaft 204 may be a solid member. According to these embodiments, the conductive element 102 may be disposed along the inner shaft 204.

Further, outer sheath 107 and the inner shaft 204 may be made of biocompatible materials such as suitable polymers or metals. Both outer sheath 107 and the inner shaft 204 may be formed from the same material, or different materials may be employed, so long as those materials are mutually compatible. In general, suitable polymeric materials include, for example, polyamide, PEBAX® (polyether block amide), polyurethane, polyethylene, nylon, and polyethylene terepthalate. Metallic materials, such as stainless steel or nitinol may also be used. Alternatively, a combination of polymeric and metallic materials may be employed as well. A suitable combination material may be a polymeric material reinforced with metallic wires braid or springs. To reduce friction, outer sheath 107 and inner shaft 204 may be coated with a suitable low-friction material, such as TEFLON®, polyetheretherketone (PEEK), polyimide, nylon, polyethylene, or other lubricious polymer coatings. These are just examples. Other materials are contemplated.

The expandable member 202 may vary in shape and/or configuration. For example, the expandable member 202 may take the form of a stent or stent-like structure. Because the expandable member 202 may be attached to the inner shaft 204 (and because the proximal end of the expandable member 202 may be disposed within the outer sheath 107), the expandable member 202 may generally take a funnel shape when the outer sheath 107 is proximally retracted. This may help in keeping the electrodes 206 at a position that is radially inward from the widest point of the expandable member 202. In other embodiments, the expandable member may have a funnel shape where the distal end is relatively larger, with the body of expandable member 202 tapering toward its proximal end. The expandable member 202 may be self-expanding, allowing it to expand as it is deployed from the distal end of outer sheath 107.

In the retracted position (FIG. 2A) and collapsed configuration, the expandable member 202 may be compressed into a cylindrical profile sufficiently small to allow the expandable member 202 to fit within the lumen of outer sheath 107. The retracted position may facilitate introduction of system 100 into a patient's vasculature, as well as subsequent navigation to a desired surgical site, such as blood vessel 200.

In the deployed position (FIG. 2B) and expanded configuration, the expandable member 202 may assume the funnel-shaped profile described above. In that profile, the radial expansion of the distal end of the expandable member 202 may be sufficient to bring that member into contact with the wall of blood vessel 200. That configuration positions electrodes 206 at a controlled distance away from the wall of the blood vessel 200. Shifting to the expanded configuration may occur by proximally retracting the outer sheath 107 relative to the inner shaft 204.

The dimensions of the expandable member 202 may be tailored to a desired application. For example, its expanded state radius may be chosen based on the expected interior diameter of blood vessel 200. Similarly, its length may be selected to suitably place the electrodes 206 at a target location within a selected renal artery.

The expandable member 202 may be made up of any suitable biocompatible polymeric or metallic material(s). Some exemplary materials that may be used are stainless steel, nitinol, Elgiloy or the like. In some embodiments, the expandable member 202 may be made by laser cutting a hypotube or sheet of material (which may be subsequently rolled into a tube-like configuration). Other methods may be used to form the expandable member 202.

The expandable member 202 may be insulated to prevent current leakage. Some exemplary methods of insulation that may be used are dip and spray coating, chemical vapor deposition, or parylene coating.

The proximal end of the expandable member 202 may attach to the distal end of the inner shaft 204 using a suitable attachment technique. Some example attachment techniques may include the use of adhesives, welding, soldering, or the like.

Electrodes 206 may be pad shaped electrodes positioned proximal to the distal end of the expandable member 202. The pad shape of the electrodes 206 may provide for a relatively large electrode surface area. This relatively large electrode surface may avoid overheating the blood near electrodes 206, which in turn may reduce clotting, electrode fouling, and/or clot embolization. In addition, the off-wall positioning of the electrodes 206 may improve deeper target tissue heating while reducing heating of the wall of the blood vessel 200. Electrodes 206 may be positioned at a tapered orientation, such that their distal ends may be near to the blood vessel 200 wall with respect to their proximal ends. Such a positioning may increase blood velocities near the electrodes 206 thereby improving heat dissipation.

The electrodes 206 may be formed integral of the expandable member 202 or may be external members that may attach to the surface of the expandable member 202. In instances where the electrodes 206 are integral to the expandable member 202, they may be formed on an electrically conductive expandable member 202 by removing insulation from the desired surface of the expandable member 202. In instances where the electrodes 206 may be external, the electrodes 206 may be made of biocompatible materials such as stainless steel or nitinol and may attach to the desired surface of the expandable member 202 by any suitable attachment means, such as welding, soldering, or use of adhesives. The external electrodes 206 may also use the electrically conductive expandable member 202 or a separate lead or power wire (not shown) that is attached to the electrodes.

In operation, as shown in FIG. 2C, the electrodes 206 may be spaced from the wall of blood vessel 200, and in an orientation that may be referred to as off-wall electrode positioning. This orientation may provide for space between the electrodes 206 and the wall of blood vessel 200, allowing fluid flow between the electrodes 206 and the blood vessel wall. Fluid flow (e.g., including flow of blood or other fluids such as water, etc.) between the electrodes 206 and the blood vessel wall may enhance heat dissipation from surrounding tissue during ablation, minimizing or preventing thermal injury to the blood vessel 200.

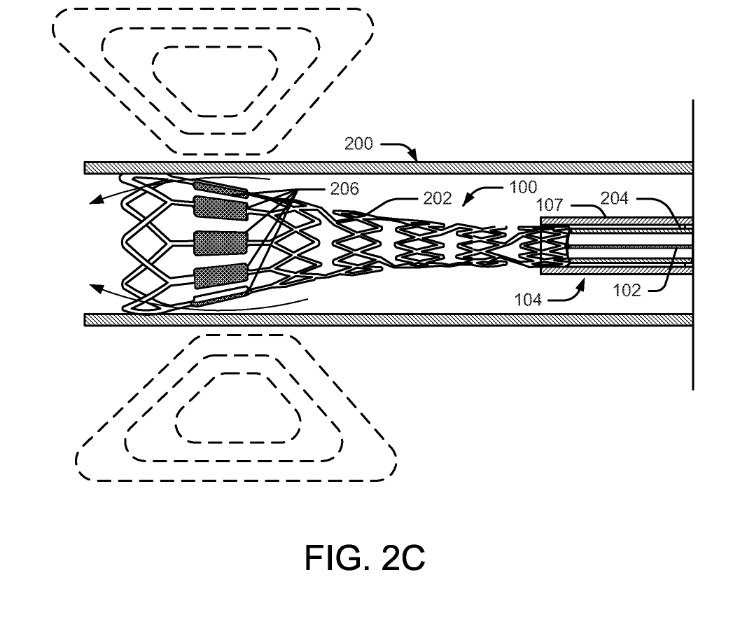

FIG. 3 depicts an alternate embodiment of the expandable member 300 similar in form and function to other expandable members disclosed herein. As shown, expandable member 300 may retain the expanded “funnel-like” shape of other embodiments. The expandable member 300 may also include additional variations. For example, the expandable member 300 may have a braided construction. In addition, the expandable member 300 may include a band-shaped electrode 302. Band-shaped electrode 302 can be utilized in other embodiments of the expandable member including those disclosed herein. As with the previous embodiment, expandable member 300 may expand or retract, assuming a funnel-like shape in the expanded (deployed) state and a compressed, cylindrical shape upon retraction. The expandable member 300 may have dimensions similar to the first embodiment, and may include additional components such as filaments, tubes, or strings to facilitate deployment or retraction.

The expandable member 300 may be made up of a wire braid of biocompatible polymeric or metallic materials for example, stainless steel, or nitinol. The wires may be either electrically conducting or non-conducting. If the expandable member 300 is electrically conducting, insulation may be applied upon it. Some exemplary methods of insulation that may be used are dip and spray coating, chemical vapor deposition, or parylene coating.

Electrode 302 may be a thin conductive membrane disposed over a portion of the expandable member 300 proximate to distal end of the expandable member 300. The electrode 302 may be disposed on either outer, inner or both surfaces of the expandable member 300. In some embodiments, the electrode 302 may include thin film segments that are separated from each other by spaces that are connected by thin film connectors such as strut pairs on a stent (and/or portions of expandable member 300). This may allow the electrode 302 to be elastic so as to collapse and expand with the expandable member 300.

The length and width of the electrode 302 may depend upon a suitable application. For example, its length may be substantially equal to the circumference of the portion of expandable member 300 where it is disposed, and its width may depend upon the size and location of the region to be treated by ablation. The electrode 302 may be made of biocompatible materials, either conducting or non-conducting materials. If conducting materials are utilized, the entire electrode 302 may function as an ablating electrode. If non-conducting materials are included, the electrode 302 may be plated with or otherwise include a conducting material that defines one or more discrete electrodes.

In operation, electrode 302 may connect to the conductive element 102 either directly or through the expandable member 300 to provide electrical energy for ablation. Additionally, the electrode 302 may partially occlude the blood vessel 200 thereby increasing blood velocity in the blood vessel 200. As discussed, increased blood velocity may increase dissipation of heat, and thus may prevent thermal injury.

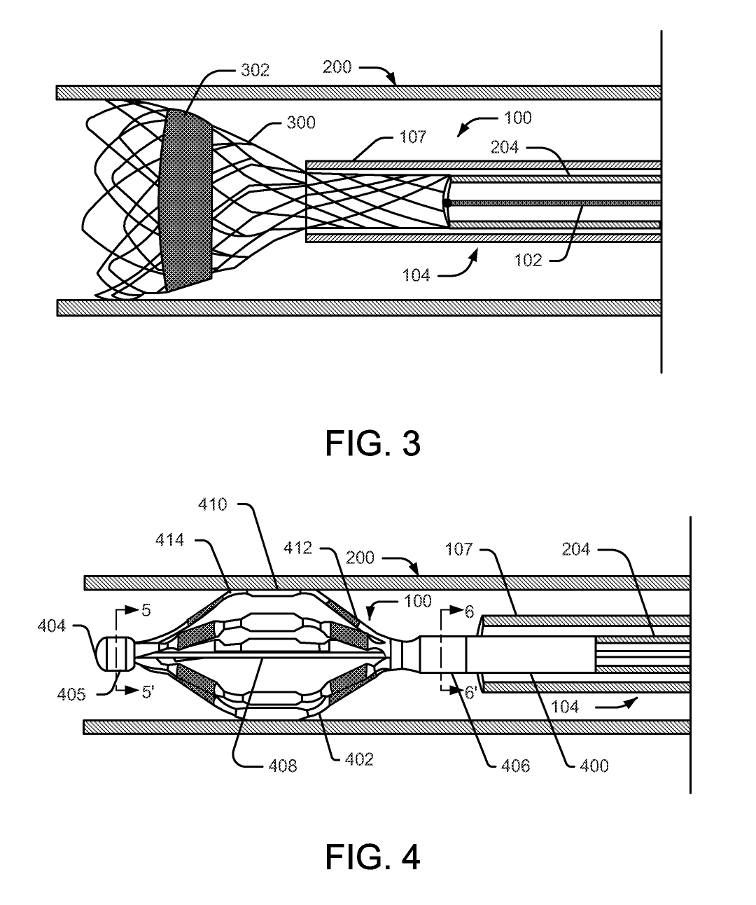

FIGS. 4-6 illustrate another alternate embodiment of the expandable member according to the present disclosure. Here, the expandable member may be an expandable basket 400, whose form may be provided by one or more struts or ribbons 402, joined at their distal ends by a distal weld ball 404 and distal hypotube 405. The proximal ends of the ribbons 402 are joined in a tubular member 406 (support hypotube), which may extend proximally to the proximal end of the system. In some embodiments, a compression resistance coil (not shown) may be disposed at the proximal end of the ribbons 402 and/or within the tubular member 406.

The basket 400 may be either symmetric, or asymmetric. For example, some ribbons 402 may be staggered from other ribbons 402. The ribbons 402 may be generally axial, or may have circumferential or spiral orientation about the longitudinal axis of the basket 400. The ribbon 402 lengths, insulation locations, and overall geometry and angles may be chosen for acceptable deployment in a range of artery sizes. Alternatively, pre-sized ribbons 402 may be used, chosen for precise deployment configuration in the size vessel being treated, for example, as shown in blood vessel 200. Each ribbon 402 may include one or more wall-contact segments 410, one or more electrode segments 412, and one or more bend segments 414. In some embodiments, the wall-contact segments 410 are positioned generally in a central region of each ribbon 402, with one bend segment 414 proximal and another bend segment 414 distal of the contact segment 410. Electrode segments 412 may be positioned between bend segments 414 and the distal and proximal ends of each ribbon 402, respectively.

A control wire 408 may provide electrical contact with electrodes 412. In other words, the control wire 408 may be used to supply current or otherwise “power” the electrodes 412. In addition, the control wire 408 may also be used to collapse and expand the basket 400. For example, the control wire 408 may be urged distally to shift or otherwise “push” the basket 400 into a collapsed configuration and the control wire 408 may be urged proximally to shift or otherwise “pull” the basket 400 into an expanded configuration. The use of such a control wire 408 that provides both power to the electrodes 412 and controlled shifting of the basket 400 may be desirable for a number of reasons. For example, the use of such a control wire 408 may help to simplify the manufacturing of the renal ablation system 104.

FIG. 5 is a cross-sectional view of the distal end portion of the nerve modulation system 100, taken on plane 5-5′ of FIG. 4. As shown, the distal ends of ribbons 402 are held between distal hypotube 405 and spacer tube 502. Spacer tube 502 may include a suitable material such as any of those materials disclosed herein such as stainless steel, a polyetherimide (e.g., ULTEM, commercially available from SABIC Innovative Plastics IP BV, Pittsfield, Mass.), or other suitable materials. If desired, ribbons 402 may be welded, brazed, or otherwise fixed in position. Additionally, control wire 408 extends into distal hypotube 405 at this location. An electrical connection (not shown) may provide contact between control wire 408 and ribbons 402.

FIGS. 6A-6B illustrate two embodiments of the nerve modulation system 100 taken on plane 6-6′ of FIG. 4. Referring to FIG. 6A, the proximal ends of ribbons 402 extend into support hypotube 406, where they are held between the support hypotube 406 and a spacer tube 602. Holes 604, formed in the sides of support hypotube 406, may be used to secure the hypotube 406 to the spacer tube 602 (e.g., via soldering). Control wire 408 runs through spacer tube 602, and may be insulated to prevent unwanted electrical contact in this portion of the device.

The embodiment of FIG. 6B substitutes two elements for the control wire 408 of FIG. 6A. Here, a control wire 606 provides the control function (e.g., shifting the basket 400 between a collapsed and an expanded configuration), and power wire 608 provides electrical power. The power wire 608 may attach to the proximal end of the basket 400. The power wire 608 may be located apart from the center of the devices, in a location such as disposed between the support hypotube 406 and spacer tube 602. The use of a distinct control wire 606 and a distinct power wire 608 may be desirable for a number of reasons. Even though such a design may include more parts, each part may be optimized for its intended function. For example, the control wire 606 may be designed so as to minimize stiffness while still being able to expand and contract the basket 400. Likewise, the power wire 608 may be designed to minimize power transmission losses to the basket by using a material like copper wire. These are just examples. Other features and/or benefits are contemplated.

Structurally, the basket 400 may be designed with various numbers of ribbons 402, for example, 2, 3, 4, 5, 6, 7, or 8, arranged circumferentially around the control wire 408 along the longitudinal axis of the basket 400. The distal portion of the basket 400 may hold the weld ball 404 attached to the control wire 408. The distal hypotube 405 may connect proximally to the weld ball 404. The distal portions of the ribbons 402 may be affixed between the distal hypotube 405 and the spacer tube 502. Similarly, the proximal portions of the ribbons 402 may attach between the support hypotube 406 and the spacer tube 602. To attach the ribbons 402, methods such as stamping, welding, or reflow soldering may be used. In some embodiments, holes 604 may be made in the support hypotube 406 to facilitate a reflow soldering process. Further, the control wire 408 may pass proximally through the center of the arrangement.

Each ribbon 402 may include preformed bend segments 414 positioned at various locations within the ribbon 402. The location of the bend segments 414 may depend upon the desired shape of the ribbon 402 after expansion of the basket 400. For example, in the present embodiment, with bend segments 414 in each ribbon 402, assumes a generally cylindrical shape upon expansion of the basket 400. It should be noted that the position and preformed shape of bend segments 414 largely determine the eventual shape of basket 400. In some embodiments, a generally cylindrical shape can be retained, with the central portion of ribbon 402 assuming a more or less bowed shape, as desired. Employment of shape memory materials, such as nitinol, may enhance the ability to achieve exact configurations to fit various applications. The ribbons 402 also include wall contact segments 410 that may contact and align with the wall of the blood vessel 200 upon expansion of the basket 400.

When basket 400 is expanded, ribbons 402 extend radially outward. Different ribbon constructions can lead to different basket shapes, as seen in the various embodiments set out herein. The embodiment illustrated in FIG. 4 may include two bend segments 414 located about ⅓ the distance from the proximal and distal ends of basket 400. Consequently, the basket expansion causes each ribbon 402 to assume a shape having a linear wall contact segment 410 lying generally parallel to the longitudinal axis of basket 400, with similarly straight electrode segments lying proximal and distal to the wall contact segment 410, each forming an obtuse angle with it and extending toward the control wire 408. Each ribbon 402 may be formed of an electroconductive material, covered with an insulative coating. A bare patch on each electrode segment 412 forms an electrode for applying ablation energy to the vessel 200. Thus, the illustrated embodiment may have two electrodes per ribbon, one on the electrode segment proximal of the wall contact segment 410 and one on the electrode segment distal of the same. The structure of this embodiment serves to position electrode segments 412 a selected distance from the wall of vessel 200.

Further, the thermal geometry of the ablation process may be modified by changing parameters such as location, length, and spacing from the artery wall; circumferential and axial spacing; angular orientation; and surface area of the electrode segments 412. Therefore, it may be noted that a person skilled in the art may alter these parameters to produce a desired heating pattern on the blood vessel 200. For example, circumferentially arranged electrode segments 412 around the basket 400 may provide for a desired heating of a circumferential target site, while maintaining the non-treated portion of the blood vessel 200 at lower temperatures to minimize vessel wall injury.

The electrode segments 412 and the wall contact segments 410 may be wider than the bend segments 414. The smaller width of the bend segments 414 may aid in bending the ribbons 402, while the larger width of the wall contact segments 410 may provide adequate support to the wall of blood vessel 200 upon expansion of basket 400. Further, wide electrode segments 412 may reduce thermal heating of the surrounding tissue, thereby reducing the risk of thermal injury to the blood vessel 200. In some embodiments, struts (not shown) may attach to the proximal and distal ends of each ribbon 402 to hold the elements of the basket 400 together and maintain the geometrical shape of the basket 400.

The control wire 408 may connect to conductive element 102 and may provide electrical energy to the basket 400. In addition, the control wire 408 may function as a control mechanism to expand or collapse the basket 400. Upon proximal retraction of the control wire 408, the basket 400 may expand, and upon distally moving the control wire 408, the basket 400 may collapse. This may provide a simple control mechanism to shift the basket 400 between the collapsed and expanded configurations. However, it may be noted that it is not the only control mechanism that may be used with the basket 400, and persons of average skill in the art may contemplate various other control mechanisms.

Various methods may be used to manufacture the basket 400. In some instances, the basket 400 may formed from a cut metal tube. The metal tube may be laser cut to form the ribbons 402 and some other structures of the basket 400, while some other structures may be attached to the basket 400 by any attachment mechanism, such as welding, soldering, stamping, or use of adhesives. In some other embodiments, each ribbon 402 may be made separately and combined in assembly to form the basket 400.

Biocompatible materials such as suitable polymers or metals may be used to form the basket 400 and its components such as, the ribbons 402. In general, suitable polymeric materials may include, for example, the polyamide, PEBAX® (polyether block amide), polyurethane, polyethylene, nylon, and polyethylene terepthalate. Metallic materials, such as stainless steel or nitinol may also be used. In addition, the basket 400 may be insulated such that only the electrode segments 412 may not have insulation. This insulation may prevent unwanted current leakages from the basket 400. Some exemplary methods of insulation that may be used are dip and spray coating, chemical vapor deposition, parylene coating or by slipping tight fitting tubing over the ribbons 402 such as using an electrically insulating shrink tubing.

In operation, similar to the previous embodiments, the basket 400 is configured to shift between a collapsed and an expanded configuration. For example, the basket 400 may rest within the outer sheath 107 in the collapsed configuration state. The outer sheath 107 may be proximally refracted to expose the basket 400. In general, the position of the basket 400 may remain stationary relative to the blood vessel 200 during expansion/deployment while the outer sheath 107 moves proximally to expose the basket 400. It should also be noted that the guide catheter 106 may serve the purpose of the outer sheath 107. In at least some of these example, the guide catheter 106 may not enter a renal artery and, instead, be positioned at the ostium of the renal artery. The basket 400, in a collapsed state, would enter the renal artery by being guided by the guide catheter 106 located at the ostium of the renal artery. Unlike the previously discussed embodiments, which are self-expanding and may expand upon deployment, the basket 400 may need a control mechanism, for example, the control wire 408 to shift into expanded and collapsed configurations. After expansion, the wall contact segments 410 may contact the wall of the blood vessel 200 to hold the basket 400 at a desired location within the blood vessel 200. In addition, the electrode segments 412 may position at a location proximate to but not contacting the wall of the blood vessel 200. After positioning of the electrode segments 412, RF ablation may be carried out to ablate renal nerves. As noted above with the previous embodiments, this process may allow for off-wall (non-contact) ablation of renal arteries within the blood vessel 200, thereby reducing the risk of inadvertent damage to the blood vessel 200 and the depth of ablation.

In some implementations, ground pads 110, as shown in FIG. 1, may be used to complete the circuit, energizing the electrode segments 412 in a unipolar manner. Alternatively, the ribbons 402 may be electrically isolated from each other, with energy applied between ribbons 402 in a bipolar manner. In another alternate embodiment, an electrical break (not shown) may be included within the wall contact segments 410 or the bend segments 414 so that the distal ends of ribbons 402 are electrically isolated from the proximal end of the ribbons 402, and the electrode segments 412 may be energized in a bipolar manner. In an alternative bipolar arrangement the individual ribbons may alternate between hot and ground, thus creating a circumferential current path instead of a basket end to basket end current path as described. The control and power element 108 may energize all electrode segments 412 simultaneously. Alternatively, single electrode segment 412, or groups of electrode segments 412, may be isolated from others, with separate control to achieve a desired balanced or unbalanced power delivery among the electrode segments 412.

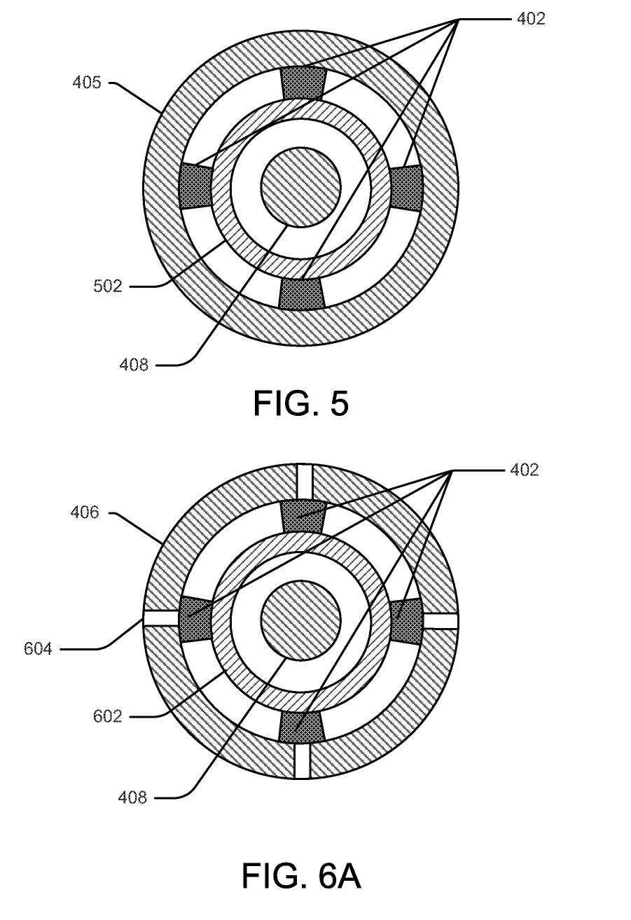

FIG. 7 illustrates another embodiment of the expandable member. This embodiment may be a basket 700 similar to the basket 400 of the FIGS. 4-6. The basket 700 may be structurally similar to the basket 400. However, unlike the segmented ribbons 402 in basket 400, the basket 700 may include ribbons 702 structured as metal strips. The basket 700 may be symmetric or asymmetric, as desired. For example, some ribbons 702 may be staggered from other ribbons 702, or uninsulated portions 706 may be arranged in a spiral pattern. The ribbons 702 may be generally axial, or may have circumferential or spiral orientation about the longitudinal axis of the basket 700. The ribbon 702 lengths, insulation locations, and overall geometry and angles may be chosen for acceptable deployment in a range of artery sizes. Alternatively, pre-sized ribbons 702 may be used, chosen for precise deployment configuration in the size vessel being treated, for example, as shown, blood vessel 200. The ribbons 702 may be kept aligned by one or more extruded profile polymer sleeves (not shown) located at the ends of the ribbons 702.

Suitable biocompatible materials known in the art along with those mentioned above for forming the basket 400 (FIG. 4) and its components may be used for making the basket 700 and its components such as the ribbons 702. Similarly, the ribbons 702 may be partially insulated by dip or spray coating, chemical vapor deposition, parylene coatings, a tight fitting tube or the like, for example an electrically insulating shrink tubing.

Each partially insulated ribbon 702 may have one or more insulated portions 704 and one or more uninsulated portions 706. The uninsulated portions 706 may be positioned proximate to the ends of the basket 700 lying radially inwards relative to the greatest radial extent of the basket 700, and the insulated portions 704 may be positioned at the greatest radial extent of the basket 700. The insulated portions 704 may be positioned at other locations if desired.

In operation, the uninsulated sections 706 may function as electrodes for RF ablation while the insulated portions 704 may contact the wall of the blood vessel 200 upon expansion of the basket 700, and thus may hold the basket 700 in position during ablation. Similar to the previous embodiment, the basket 700 may first deploy and then be actively expanded by refraction of the control wire 408 by an operator. During expansion, the ribbons 702 may flex radially outward to expand the basket 700. After expansion, the insulated sections 704 may contact the wall of blood vessel 200 and may hold the basket 700 firmly in position. Further, this process places the uninsulated sections 706 at a controlled distance away from the blood vessel 200 wall. Then, RF ablation may be carried out using the uninsulated sections 706. As discussed earlier, various combinations of uninsulated sections 706 (electrodes) may be used depending upon desired effects. Further, heating geometry of the target can be modified by changing various parameters such as location, length, or surface area of the uninsulated sections 706.

In other implementations, ground pads 110, as shown in FIG. 1, may be used to complete the circuit, energizing the electrodes (uninsulated sections 706) in a unipolar manner. Alternatively, the ribbons 702 may be electrically isolated from each other, and energized between ribbons 702 in a bipolar manner. In another alternate embodiment, an electrical break (not shown) may be included under the insulated portion 704 so that the distal end of the ribbons 702 are electrically isolated from the proximal end of the ribbons 702, and the electrodes 706 may be energized in a bipolar manner. The control and power element 108 may energize all electrodes 706 simultaneously. Alternatively, single electrode 706, or groups of electrodes 706, may be isolated from others, with separate control to achieve a desired balanced or unbalanced power delivery among the electrodes 706.

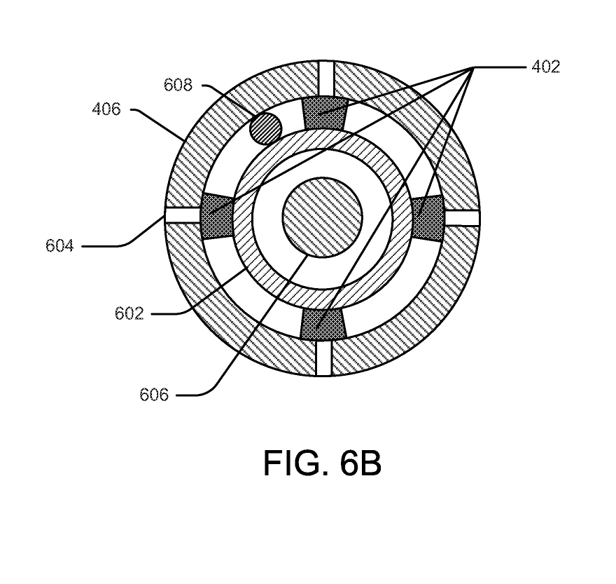

FIG. 8 illustrates yet another embodiment of the expandable member, which may take the form of a basket 800 similar to basket 700 of FIG. 7. Here, basket 800 may include partially insulated ribbons 802 having insulated portions 804 (wall-contact) and uninsulated portions 806. However, as shown, the ribbons 802 may be shaped and sized to bend substantially more than ribbons 702 (FIG. 7) such that the two opposing halves of each ribbon 802 may lie at acute angles with respect to each other. This orientation may provide a shorter basket 800, and may facilitate improved ablation of target tissue and safety.

Suitable biocompatible materials known in the art along with those mentioned above, for forming the basket 700 (FIG. 7) and its components may be used for making the basket 800 and its components such as the ribbons 802. Similar to ribbons 702, the ribbons 802 may be kept aligned by an extruded profile polymer sleeve (not shown), and may be insulated by any of the methods mentioned above. In some embodiments, the sides of the insulated portions 804, which are oriented towards the center of the basket 800, may be uninsulated, since these sides of the insulated portions 804 may not contact the artery wall. This additional space may be used to increase surface area of the uninsulated portion 806 and/or to provide an electrode positioning closer to the artery wall.

In operation, basket 800 may function similar to the basket 700. The uninsulated portions 806 may function as electrodes for RF ablation while the insulated portions 804 may contact the wall of the blood vessel 200 upon expansion of the basket 800 holding the basket 800 in position during RF ablation. As described above, different arrangement of the electrodes (uninsulated portions 806) and different electrical configurations may be used for conducting RF ablation.

In some alternate embodiments, rather than using non-contact electrodes 806 as shown, multiple wall-contact electrodes (not shown) may be formed in a similar manner, but leaving the central sections of the ribbons 802 that contact the wall uninsulated; insulation may be used to cover other portions of the ribbon 802.

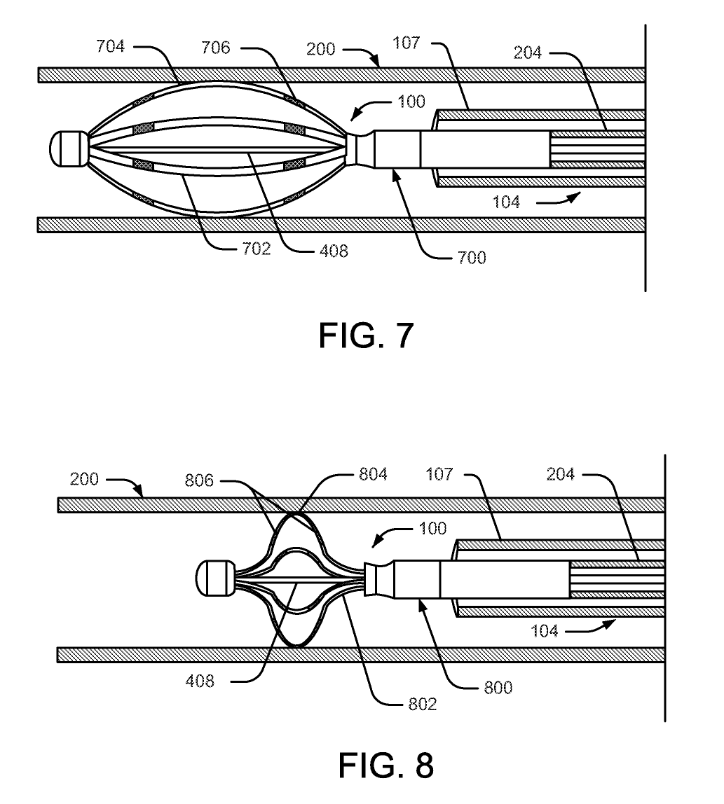

FIG. 9 illustrates another embodiment of the expandable member 900. The expandable member 900 may include multiple baskets 902 (902A, 902B . . . ), each similar to the basket 800 shown in the previous embodiment. The figure depicts two baskets 902A and 902B but it will be understood that any suitable number of baskets 902 may be employed, connected in series. The structure of the expandable member 900 containing the two baskets 902A, 902B may provide increased stability within the blood vessel 200. This structure of the expandable member 900 may thus, aid in maintaining the expandable member 900 aligned in the blood vessel 200 to ensure the desired position of the electrode(s) 806. A bushing 904 may be added between the baskets 902A, 902B to align the baskets 902 with the inner shaft 204. The bushing 904 may be insulated or not, depending on the desired electrode 806 surface area and location. In some embodiments, the distal basket 902A may be smaller in dimensions than the proximal basket 902B. This arrangement may facilitate the use of the expandable member 900 in a tapered vessel.

The electrodes 806 may be used in various arrangements or patterns to ablate renal nerves effectively. For example, the inner portions of the baskets 902A and 902B may include electrodes 806, or the outer portions may contain the electrodes 806. Alternatively, in some embodiments, the expandable member 900 may include three baskets (not shown). The baskets at the distal and proximal ends may be insulated for alignment and the center basket may include electrodes 806.

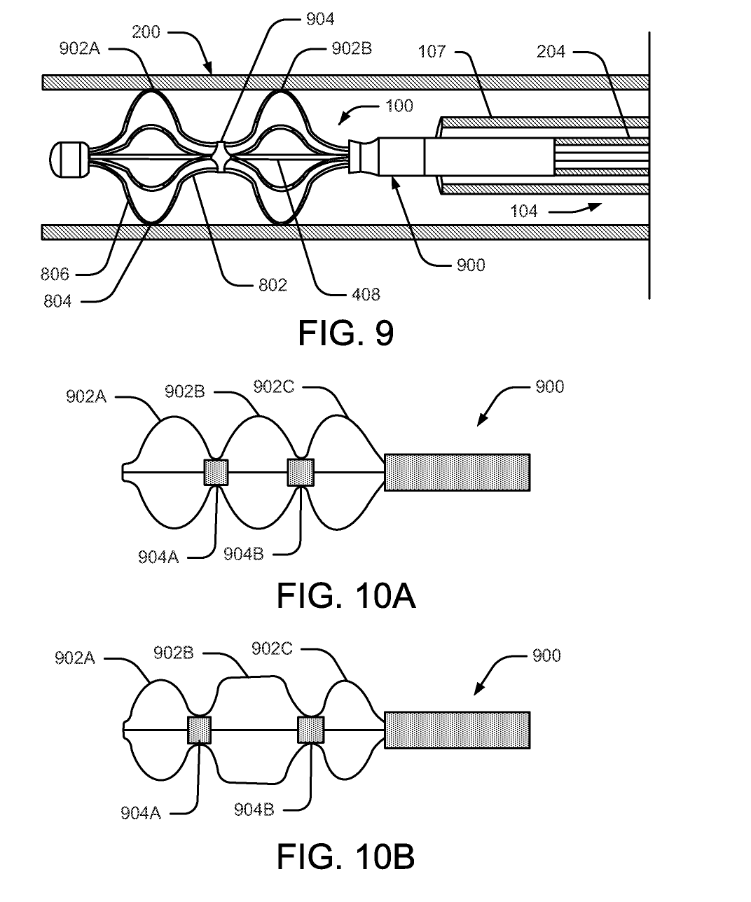

In general, the expandable member 900 may be designed with various arrangements of the baskets 902, and various orientations, shapes and configuration of ribbons 802. For example, FIGS. 10A-10C depict simplified diagrams of expandable member 900, representing variations in the features mentioned above. FIG. 10A depicts three identical baskets 902A, 902B, and 902C arranged in series with two intermediate bushings 904A, 904B. FIG. 10B illustrates three baskets 902A, 902B, 902C and intermediate bushings 904A, 904B, wherein the central basket 902B may be broader than the end baskets 902A and 902C. FIG. 10C exhibits two identical baskets 902A and 902B separated by the bushing 904. These are just examples and many other suitable arrangements for expandable member 900 may be contemplated.

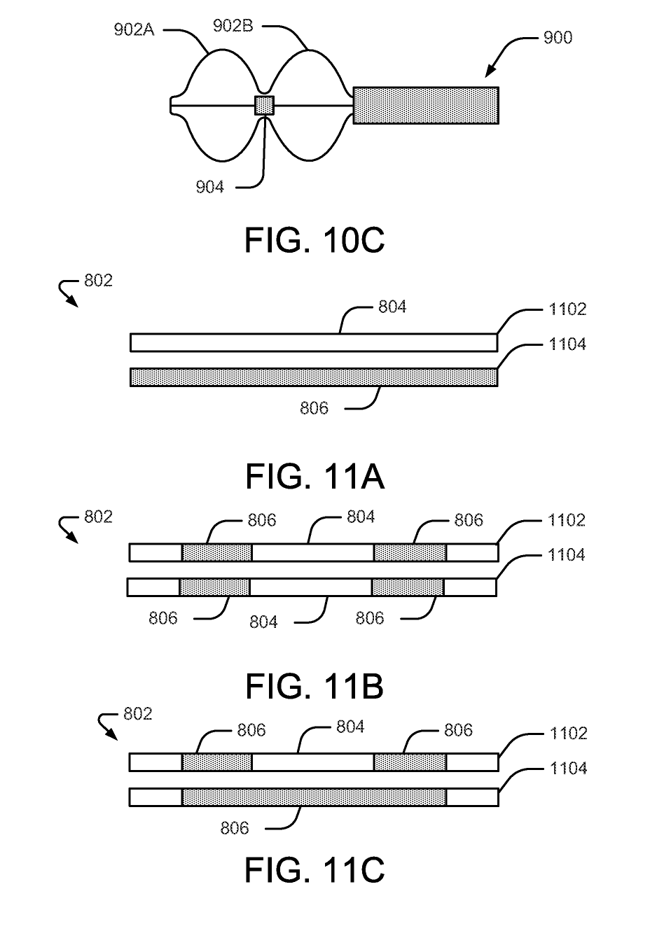

In addition to variation in ribbon 802 shape and orientation, various insulation configurations may be used. For example, FIGS. 11A-11E depict different insulation configurations of ribbons 802. As shown in FIG. 11A, the entire outer portion 1102 of the ribbon 802 may be insulated to protect against electrical contact with the blood vessel 200 wall, while the entire inner portion 1104 of the ribbon 802 may be kept bare; this may be accomplished by applying an insulating material to the outer portion 1102, or applying an insulating material to all sides of the ribbon 802 and subsequently removing the insulation from the inner portion 1104, or by forming a layered base material from which the ribbons 802 may be cut. Alternatively, as shown in FIGS. 11B-11C, insulating tubing, such as shrink tubing may be slid over selected portions of the ribbons 802. Selected portions of the tubing may be removed after shrinking the tubing onto the ribbons 802; for example, segments of shrink tube may be completely slid over and then shrunk onto ribbons 802. Portions could then be trimmed off to form the uninsulated electrode portions 806. In other examples, as shown in FIGS. 11D-11E, selected portions may be removed, leaving “belt loop” structures 1106 to secure the insulating tubing in place on the ribbon 802.

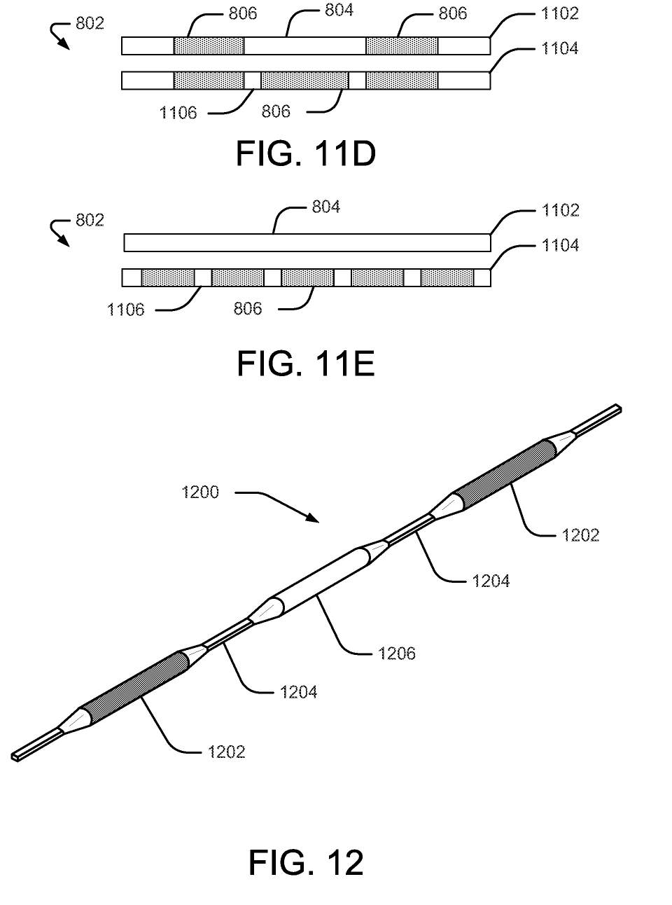

In addition, portions of the ribbons that contact the artery wall and are insulated, the sides or edges of the ribbons may be insulated. Flat wire ribbons, or round wires, or other profiles can be used for the ribbons 802. For example, FIG. 12 depicts an alternate embodiment of a ribbon 1200. As described with the previous embodiments, one or more baskets may be employed as an expandable member. The ribbons within the discussed baskets were merely exemplary and other embodiments of the ribbons may be used within such baskets (expandable member). The present embodiment is an alternative ribbon 1200 that may be used within the discussed baskets (basket 400, basket 700, basket 800, and baskets 902).

The ribbon 1200 may include one or more electrode portions 1202, bend portions 1204, and wall contact portions 1206. The electrode portions 1202 may have a substantially round profile, while the bend portions 1204 may have a substantially flat profile. The wall contact portions 1206 may have any profile that may not harm the wall of the blood vessel 200. In some embodiments, as shown, the wall contact portions 1206 may have a substantially round profile.

In cases where the electrodes are flat or have sharp corners, current concentrations may be higher at the corners and may not spread evenly on the electrode surface. This uneven current concentration may lead to uneven heating of the blood vessel 200, which may cause inadvertent damage to the blood vessel 200 tissue. The round profile of the electrode portions 1202 may allow for even spread of current concentrations on the electrode surface, which may improve the heating geometry of the blood vessel 200 during ablation. In addition, the flat profile of the bend portions 1204 may allow them to bend easily upon application of force. The bend portions 1204 may facilitate expansion and contraction of the basket.

The ribbon 1200 may be formed from a round wire with the flat bend portions 1204 created by any of the various machining or forming operations known in the art. Alternatively, the ribbon 1200 may be formed by cutting a hypotube. The hypotube may be laser cut to form the electrode, bend, and wall contact portions 1202, 1204, and 1206. A post processing operation may be used to thin selected regions to get preferential bending at those regions.

The ribbon 1200 may be partially insulated. The bend portions 1204 and the wall contact portions 1206 may be insulated to prevent current leakages, while the electrode portions 1202 may be uninsulated to conduct ablation. Some exemplary methods of insulation that may be used are dip and spray coating, chemical vapor deposition, parylene coating or by slipping tight fitting tubing over the ribbon 1200 such as using an electrically insulating shrink tubing.

Alternatively, electrode segments 412 in ribbons 402 of basket 400 (FIG. 4) may, uninsulated portions 706 in ribbons 702 of basket 700 (FIG. 7) may, and uninsulated portions 806 in ribbons 802 of basket 800 (FIG. 8) may have a round profile to evenly spread current on the electrode surface, which may improve the heating geometry of the blood vessel 200 at the time of ablation. Similarly, the bend segments 414 and the insulated portions 704 may have substantially flat profile to improve bending.

An exemplary method for renal nerve ablation using the system 100 may be illustrated using FIG. 1 and FIGS. 2A-2C. Referring to FIG. 1, an operator may introduce the outer sheath 107 within a patient's vasculature through a guide catheter 106. Further, the operator may maneuver the outer sheath 107 to the desired location for renal nerve ablation within the vasculature of the patient. Referring to FIGS. 2A-2B, after reaching the desired location within the desired blood vessel 200, the operator proximally retracts the outer sheath 107 to shift the renal ablation system 104 from the initial retracted position to the deployed position. After expansion, the distal portion of the expandable member 202 may contact the wall of the blood vessel 200 and position the electrodes 206 at a controlled location from the wall of blood vessel 200 at the target site. After positioning the electrodes 206, the operator may use the control and power element 108 to transmit RF electrical energy to electrodes 206 through conductive element 102. The circumferentially placed electrodes 206 may ablate renal nerves perivascularly. After conducting ablation, the operator may advance the outer sheath 107 over the expandable member 202 and remove the renal ablation system 104 from the patient.

The alternative embodiments disclosed herein may essentially follow a similar method of use with some additional or different steps. For example, the basket 400, basket 700, basket 800, and expandable member 900 may require an additional step for shifting into expanded configuration from collapsed configuration upon deployment as they are not self-expanding. Referring to FIG. 4, in case of basket 400, an exemplary additional step may be to push the control wire 408 forward to expand the basket 400 after bringing the renal ablation system 104 in the deployed position.

The materials that can be used for the various devices and/or systems (and/or components thereof) disclosed herein may include those commonly associated with medical devices. For example, the devices, systems, and/or components disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions of the devices, systems, and/or components disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices disclosed herein in determining their location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the devices/systems to achieve the same result.

In some embodiments, a degree of Magnetic Resonance Imaging (MRI) compatibility may be incorporated into the devices, systems, and/or components disclosed herein. For example, the devices/systems may be made of a material that does not substantially distort the image and create substantial artifacts (i.e., gaps in the image). Certain ferromagnetic materials, for example, may not be suitable because they may create artifacts in an MRI image. Devices/systems may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Although the embodiments described above have been set out in connection with a renal nerve ablation catheter, those of skill in the art will understand that the principles set out there can be applied to any catheter or endoscopic device where it is deemed advantageous to perivascularly ablate nerve cells. Conversely, constructional details, including manufacturing techniques and materials, are well within the understanding of those of skill in the art and have not been set out in any detail here. These and other modifications and variations may well be within the scope of the present disclosure and can be envisioned and implemented by those of skill in the art.

Other embodiments of the present disclosure will be apparent to those skilled in the art from consideration of the specification and practice of the embodiments disclosed herein. It is intended that the specification and examples be considered as exemplary only, and departure in form and detail may be made without departing from the scope and spirit of the present disclosure as described in the following claims.

What is claimed is:

1. A medical device for ablating nerves perivascularly, the medical device comprising:
an expandable frame slidably disposed within a catheter shaft, the expandable frame being configured to shift between a collapsed configuration and an expanded configuration; and
one or more electrodes disposed on a surface of the expandable frame,
wherein the one or more electrodes are disposed radially inward relative to the greatest radial extent of the expandable frame when the expandable frame is in the expanded configuration.

2. The medical device of claim 1, wherein the expandable frame is self-expanding.

3. The medical device of claim 1, wherein the expandable frame is actively expanded and collapsed by a control mechanism.

4. The medical device of claim 1, wherein the expandable frame includes a plurality of struts.

5. The medical device of claim 1, wherein the expandable frame is attached to an inner shaft, and proximal retraction of the outer sheath at least partially deploys the expandable frame.

6. The medical device of claim 1, wherein the expandable frame includes a stent-like frame and the electrodes are disposed within the stent-like frame.

7. The medical device of claim 1, wherein the expandable frame is a stent-like frame, and wherein the electrodes are defined by a conductive band disposed about the stent-like frame.

8. The medical device of claim 1, wherein the expandable frame includes a basket comprising one or more conductive ribbons.

9. The medical device of claim 8, wherein each ribbon includes:
an insulated wall contact alignment segment, positioned in the expanded configuration at the greatest radial extent of the expandable frame;
an uninsulated electrode segment, positioned near an end of the ribbon and lying radially inwards relative to the greatest radial extent of the expandable frame; and
a joining segment joining the wall contact alignment segment and the uninsulated electrode segment.

10. The medical device of claim 9, wherein the uninsulated electrode segment has a substantially round profile and the joining segment has a substantially flat profile.

11. The medical device of claim 8, wherein each ribbon includes:
one or more uninsulated portions positioned near an end of the expandable frame and being disposed radially inward relative to the greatest radial extent of the expandable frame, and
one or more insulated portions disposed at a position adjacent to the greatest radial extent of the expandable frame when the expandable frame is in the expanded configuration.

12. The medical device of claim 11, wherein the uninsulated portions have a substantially round profile and the insulated portions have a substantially flat profile.

13. A medical apparatus for ablating nerves perivascularly, the apparatus comprising:
a catheter shaft having a proximal end, a distal end, and a lumen extending from the proximal end to the distal end;
an expandable member slidably disposed within the catheter shaft; and
a control mechanism to shift the expandable member between a collapsed configuration and an expanded configuration,
wherein the expandable member includes one or more electrodes disposed on a surface of the expandable member and positioned radially inward relative to the greatest radial extent of the expandable member.

14. The medical apparatus of claim 13, wherein the expandable member includes a stent-like frame, and wherein the electrodes are disposed within the stent-like frame.

15. The medical apparatus of claim 13, wherein the expandable member includes a stent-like frame and wherein the one or more electrodes include a conductive band disposed above the stent-like frame.

16. The medical apparatus of claim 13, wherein the expandable member is a basket including conductive ribbons.

17. The medical apparatus of claim 16, wherein each ribbon includes:
a wall contact alignment segment, positioned at the greatest radial extent of the expandable member when the expandable member is in the expanded configuration;
one or more electrode segments spaced from the wall contact alignment segment and lying radially inwards relative to the greatest radial extent of the expandable member; and
bend segments having substantially flat profile, joining the wall contact alignment segment and the electrode segments.

18. The medical apparatus of claim 16, wherein each ribbon includes:
one or more uninsulated portions having a substantially round profile positioned proximate to an end of the expandable member and lying radially inwards relative to the greatest radial extent of the expandable member, and
one or more insulated portions have a substantially flat profile and are positioned at the greatest radial extent of the expandable member when the expandable member is in the expanded configuration.

19. The medical apparatus of claim 16, wherein the control mechanism includes a pull wire.

20. A method for ablating nerves perivascularly, the method comprising:
providing a medical device, the medical device comprising:
an expandable frame slidably disposed within a catheter shaft, the expandable frame being configured to shift between a collapsed configuration and an expanded configuration, and
one or more electrodes disposed on a surface of the expandable frame,
wherein the one or more electrodes are disposed radially inward relative to the greatest radial extent of the expandable frame when the expandable frame is in the expanded configuration;
advancing the medical device through a body lumen to a position adjacent to an area of interest;
shifting the expandable frame from the collapsed configuration to the expanded configuration; and
activating at least some of the one or more electrodes.
